(19) United States
(12) Patent Application Publication
(10) Pub. No.: US 2007/0255412 A1
(43) Pub. Date: Nov. 1, 2007

(54) Prosthetic device

(76) Inventors: Binyamin Hajaj, Plantation, FL (US); Rony Abovitz, Hollywood, FL (US); Wallace G. Sawyer, Gainesville, FL (US)

Correspondence Address: EDWARDS ANGELL PALMER & DODGE LLP, P.O. BOX 55874, BOSTON, MA, 02205, US

(21) Appl. No.: 11/786,929

(22) Filed: Apr. 13, 2007

Related U.S. Application Data
(60) Provisional application No. 60/792,997, filed on Apr. 18, 2006.

(51) Int. Cl. A61F 2/02

(52) U.S. Cl. 623/17.11; 623/23.76; 623/901; 623/20.17

(57) ABSTRACT

A prosthetic device includes a first component and a second component. The first component of the prosthetic device includes a polymer composite configured as an articular surface and having a wear rate of less than approximately 10⁻⁷ mm³/Nm. The polymer composite includes a first polymer and a second polymer. The first polymer comprises at least 10 weight percent of the polymer composite. The second component of the prosthetic device is configured to be implanted on a bone and includes a region of porosity that is customized for bone in-growth in a specific joint of a patient.

### CROSS REFERENCE TO RELATED APPLICATION

This application claims priority to and the benefit of Provisional U.S. Application Ser. No. 60/792,997, filed Apr. 18, 2006, the entire contents of which are incorporated herein by reference.

### TECHNICAL FIELD

The invention relates to prosthetic devices and related methods, and also to prosthetic devices for use in orthopedic joint replacement.

### BACKGROUND INFORMATION

Conventional prosthetic implants for partial or complete replacement of diseased and/or damaged joints are typically implanted on a bone and include bearing surfaces that replace the bone's natural bearing surfaces. For example, a conventional implant for a total knee replacement typically includes (a) a femoral component that is implanted on the distal end of the femur and replaces the bearing surfaces of the femur and (b) a tibial component that is implanted on the proximal end of the tibia and replaces the bearing surfaces of the tibia. In operation, the bearing surfaces of the femoral component articulate against the bearing surfaces of the tibial component. A total knee implant may also include a patellar component to replace the undersurface of the patella so that the patella slides upon the central portion of the femoral component.

In a conventional total knee replacement, the femoral component is typically made of a biocompatible metal, such as a cobalt chromium alloy, a titanium alloy, a zirconium alloy, or stainless steel, and/or may be made of a strong ceramic, such as an alumina or zirconia-based ceramic. The tibial component may include a metallic portion (or tibial tray) that is affixed to the bone and an ultra-high molecular weight polyethylene (“UHMWPE”) portion (or tibial insert) that forms the bearing surfaces of the tibial component. Alternatively, the tibial component may be made entirely of polyethylene. The patellar component typically includes a polyethylene bearing surface that is cemented to the back side of the patella directly or through a metal interface.

A problem with conventional implants is that an implant may not last for the lifetime of a patient. One variable impacting implant longevity is the wear rate of the articulating surfaces. The rate of wear is influenced by various factors, including the surface finish of the articulating surfaces, the coefficient of friction between the surfaces, the stresses generated at the surfaces, materials compatibility, and other factors. To reduce the rate of wear in conventional implants, metallic bearing surfaces (such as the bearing surface of the femoral component in a total knee implant) are typically polished to a very fine mirrored finish and designed with a sufficient degree of conformity to reduce contact stresses while allowing sufficient laxity to permit free movement.

The wear performance of conventional implants, however, is not optimal. For example, conventional levels of bearing surface wear increase dimensional tolerances (or play) between components and create particulate matter that can contaminate the joint and trigger adverse responses in surrounding tissue. As a result, accelerated degeneration of the bone and loosening of the implant may occur.

Another disadvantage of conventional implants is that such implants may not remain sufficiently fixed to the bone after implantation. For example, in applications where implants are cemented to bone, large loads transferred through the implant can cause the mechanical bond between the implant and the bone to degrade. In cementless applications, fixation typically relies on a coating or a textured surface that enables bone to grow into the implant. Conventional coatings and textured surfaces, however, may only permit in-growth on or near a surface of the implant. Thus, in-growth does not penetrate deeply into the implant component. As a result, implant longevity is not maximized.

Another major disadvantage of conventional implants is that such implants do not sufficiently reproduce the elasticity and structure of a healthy joint. For example, the polyethylene portion of a conventional implant (e.g., the tibial insert) does not sufficiently simulate the response of natural cartilage and meniscus to tensile, compressive, and shear forces. Similarly, the portion of a conventional implant that is fixed to bone (e.g., the tibial tray) does not simulate the natural elastic properties of the bone. As a result, undesirable stress concentrations or stress shielding may develop in the prosthetic joint leading to loosening of the implant, which in turn results in reduced implant life.

In light of the foregoing, a need exists for prosthetic devices with improved wear resistance, fixation, and material properties that are closer to those of a healthy bone.

### SUMMARY OF THE INVENTION

An aspect of the present invention relates to a prosthetic device. The prosthetic device includes a first component and a second component. The first component of the prosthetic device includes a polymer composite configured as an articular surface and having a wear rate of less than approximately 10⁻⁷ mm³/Nm. The polymer composite includes a first polymer and a second polymer. The first polymer comprises at least 10 weight percent of the polymer composite. The second component of the prosthetic device is configured to be implanted on a bone and includes a region of porosity that is customized for bone in-growth in a specific joint of a patient.

Another aspect of the present invention relates to a prosthetic device for implantation in a patient. The prosthetic device includes a bearing surface comprising a polymer composite. The polymer composite includes a first polymer and a second polymer and has a wear rate that is less than approximately 10⁻⁷ mm³/Nm. The first polymer is at least 10 weight percent of the polymer composite. The prosthetic device also includes a fixation portion that includes a plurality of cavities. The volume percent of the cavities is determined based on one or more aspects of a specific joint of the patient.

Yet another aspect of the present invention relates to a method of making a prosthetic device. The method includes providing a polymer composite that includes a first polymer and a second polymer; providing a porous material having a porosity that is determined based on characteristics of a bone of a specific joint of a patient; forming an articular surface from the polymer composite; forming a fixation component configured to be implanted on the bone from the porous material; and connecting the articular surface to the fixation component. The first polymer comprises at least 10 weight percent of the polymer composite, and a wear rate of the polymer composite is less than approximately 10⁻⁷ mm³/Nm.

### BRIEF DESCRIPTION OF THE DRAWINGS

The accompanying drawings, which are incorporated in and constitute a part of this specification, illustrate embodiments of the invention and together with the description serve to explain principles of the invention.

FIG. 1 is a perspective view of an embodiment of a prosthetic device according to the present invention.

FIG. 2 is a perspective view of an embodiment of a region of porosity of the prosthetic device of FIG. 1.

FIG. 3 is a perspective view of an embodiment of a region of porosity of the prosthetic device of FIG. 1 showing a porous substrate having a random cellular structure.

FIG. 4 is a top view of an embodiment of a region of porosity of the prosthetic device of FIG. 1 showing a porous substrate having a predetermined cellular structure.

FIG. 5 is a cross sectional view of a portion of the substrate of FIG. 3.

FIG. 6 is a side elevation view of an embodiment of a region of porosity of the prosthetic device of FIG. 1 showing a plurality of layers.

FIG. 7 is a top view of an embodiment of a layer of FIG. 6.

FIG. 8 is a cross sectional side elevation view of an embodiment of the region of porosity of FIG. 6 showing a random cellular structure.

FIG. 9 is a cross sectional side elevation view of an embodiment of the region of porosity of FIG. 6 showing a predetermined cellular structure.

FIG. 10 is a cross sectional view of an embodiment of a fixation component of the prosthetic device of FIG. 1 showing a coating.

FIG. 11 is a perspective view of an embodiment of the coating of FIG. 10 showing a sintered bead coating.

FIG. 12 is a perspective view of an embodiment of the coating of FIG. 10 showing a mesh coating.

FIG. 13 is a cross sectional view of an embodiment of a fixation portion of the prosthetic device of FIG. 1 showing a textured surface having a coating.

FIG. 14 is a perspective view of an embodiment of the prosthetic device of FIG. 1 showing a receptacle for receipt of a graft.

FIG. 15 is a cross sectional view of an embodiment of a region of porosity of the prosthetic device of FIG. 1 showing a barrier layer.

FIG. 16 is a cross sectional side elevation view of an embodiment of a second component of the prosthetic device of FIG. 1.

FIG. 17 is a top view of an embodiment of a prosthetic device according to the present invention.

FIG. 18 is a block diagram of an embodiment of a method of making a prosthetic device according to the present invention.

FIG. 19, like FIG. 16, is a cross sectional side elevation view of an embodiment of a second component of the prosthetic device of FIG. 1, but FIG. 19 also depicts natural bone and customization of a region of porosity of the second component.

### DESCRIPTION

Certain embodiments according to the invention are illustrated in the drawings. An effort has been made to use the same or like reference numbers throughout the drawings to refer to the same or like parts.

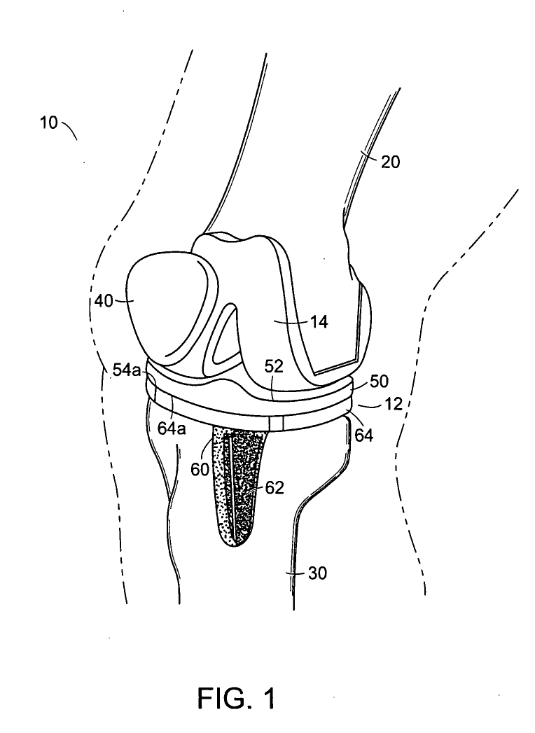

FIG. 1 is a diagram of a knee joint that includes an embodiment of a prosthetic device 10 according to the present invention. The diagram shows a lower end of a femur 20, an upper end of a tibia 30, and a patella 40. The prosthetic device 10 includes a first implant 12 and may optionally include a second implant 14. The first implant 12 includes a first component 50 and a second component 60.

The design of the prosthetic device 10 may be any design (known or future) suitable for use as an orthopedic implant. For example, the prosthetic device 10 may be any orthopedic implant, such as a knee, hip, ankle, elbow, wrist, or spine implant. The prosthetic device 10 may be a total joint implant (e.g., a total knee or hip implant), a partial joint implant (e.g., a unicondylar knee implant), or a local defect implant (e.g., a bone plug, local cartilage defect implant). According to one embodiment, the prosthetic device 10 is a total knee implant (shown in FIG. 1). In this embodiment, the first implant 12 is a tibial component, and the second implant 14 is a femoral component. The first and second components 50 and 60 of the first implant 12 comprise a tibial insert and a tibial tray, respectively. During operation of the knee joint, the second implant 14 bears and articulates against the first component 50. In another embodiment (not shown), the prosthetic device 10 may be a total hip implant. In this embodiment, the first implant 12 is a femoral component, and the second implant 14 is an acetabular component. The first and second components 50 and 60 of the first implant 12 comprise a femoral stem and femoral head, respectively. During operation of the hip joint, the acetabular component bears and articulates against the femoral head, providing a significant improvement in wear performance.

The first component 50 of the prosthetic device 10 can be disposed on the second component 60 and configured to be secured to the second component 60. For example, as shown in FIG. 1, the first component 50 may include a surface 54a, and the second component 60 may include a corresponding surface 64a. The surfaces 54a and 64a may be interconnected, for example, via a mechanical locking mechanism (e.g., a screw, press fit, dovetail joint, etc.). Alternatively, the first component 50 and the second component 60 may be integral (e.g., bonded together, molded together, adhered together etc.) with each other such that the first component 50 and the second component 60 are unable to be separated without destroying or compromising all or a portion of the first component 50 and/or the second component 60.

As shown in FIG. 1, the first component 50 includes a surface 52 that is configured as an articular surface (or bearing surface) and is designed to replace the natural surfaces of the tibia. The surface 52 may also be adapted in any known manner for use with cellular and/or orthobiologic implants. For example, the surface 52 may include at least one receptacle 51 (shown in FIG. 14) for receipt of a graft, such as a plug of cartilage cells or stem cells.

In a preferred embodiment, the first component 50 is formed either partially or entirely of a polymer composite as described in U.S. patent application Ser. No. 10/914,615, filed Aug. 9, 2004, Pub. No. US 2006/0030681; U.S. patent application Ser. No. 11/140,775, filed May 31, 2005, Pub. No. US 2006/0029795; and/or International Application No. PCT/US2005/028234, filed Aug. 9, 2005, Pub. No. WO 2006/020619, each of which is hereby incorporated by reference herein in its entirety. As described in the above-referenced patent applications, an exemplary embodiment of the polymer composite is made of particles of PTFE (a first polymer) and PEEK (a second polymer). The PTFE and PEEK particles may be formed, for example, in an interpenetrated network structure exhibiting nano-scale networking of the particles as described in Pub. No. US 2006/0020681, Pub. No. US 2006/0029792, Pub. No. WO 2006/020619, and/or “A low friction and ultra low wear rate PEEK/PTFE composite” by David L. Burris and W. Gregory Sawyer, WEAR 261 (2006) 410-418, each of which is incorporated by reference herein in its entirety. In another embodiment, the polymer composite is made of Nylon 6 (e.g., ZYTEL® ST801A NC010A manufactured by DuPont) and ultra high molecular weight polyethylene (e.g., UHMWPE GUR® 1020 manufactured by Ticona). Although the Nylon 6 and the UHMWPE can be either the first polymer or the second polymer, in a preferred embodiment, the first polymer is UHMWPE and the second polymer is Nylon 6. As with the PEEK and PTFE particles, the Nylon 6 and UHMWPE particles may be formed in an interpenetrated network structure. Particle ratios in the polymer composite can be fixed (constant) or variable (non-constant or graded) in the range of 0-100% (and vice versa) across the length, width, and depth of the polymer composite. A graded composition results in a polymer composite having a range of material properties (i.e., material properties that vary based on location within the polymer composite). Thus, by specifying the degree of polymer grading in each portion of the first component 50, the material properties of the first component 50 may be tuned or optimized for a particular application. For example, optimization of the effective modulus of elasticity across the bearing surface 52 can promote substantially uniform wear and, more significantly, substantially uniform stress distribution to the bone thereby increasing longevity of the prosthetic device 10. Similarly, the polymer composite can be graded (e.g., as described in the above referenced Pub. No. 2006/0029795) so that the elastic properties of the first component 50 effectively replicate the elastic properties of natural tissue (such as cartilage or an intervertebral disc) thereby enhancing performance of the prosthetic device 10.

Although the polymer composite preferably comprises a PTFE/PEEK composition or a UHMWPE/Nylon 6 composition, the first and second polymers may be selected from any of various polymers. For example, the first polymer may be PTFE, ultra-high molecular weight polyethylene (“UHMWPE”), or a nylon. In an exemplary embodiment, the first polymer is selected to promote articulation (e.g., reduced friction, reduced wear) between the first component 50 and the second implant 14. The second polymer may be, for example, polyetheretherketone (PEEK), polyaryletherketone (PAEK), polyimide, nylon, polycarbonate, acrylonitrile, or acrylonitrile butadiene styrene (ABS). Preferably, the first polymer comprises at least 10 weight percent of the polymer composite, and the polymer composite has a wear rate of less than approximately 10⁻⁷ mm³/Nm. The second polymer may comprise between about 10 weight percent and about 90 weight percent of the polymer composite. In one embodiment, the second polymer comprises about 50 weight percent or less of the polymer composite. Methods for producing the polymer composite are detailed in the above referenced Pub. No. US 2006/0030681, Pub. No. US 2006/0029795, and/or Pub. No. WO 2006/020619.

As explained above, the first component 50 may be made entirely of the above-described polymer composite. Alternatively, the first component 50 may be a combination of the polymer composite and any other material suitable for use in orthopedic implant applications. For example, the first component 50 may include a biocompatible metal (e.g., a cobalt-chromium alloy, a titanium alloy, stainless steel, or tantalum); a strong ceramic (e.g., an alumina or zirconia-based ceramic); one or more high performance polymers (e.g., UHMWPE); and/or a polymer composite. In a preferred embodiment, at least the articulation surface 52 of the first component 50 is formed of the polymer composite.

Similarly, the implant 14 (which articulates against the surface 52 of the implant 12) may be made of, for example, a biocompatible metal (e.g., a cobalt-chromium alloy, a titanium alloy, or stainless steel); a strong ceramic (e.g., an alumina or zirconia-based ceramic); one or more high performance polymers (e.g., UHMWPE); and/or a polymer composite such as, for example, the polymer composite described in the above referenced Pub. No. US 2006/0030681, Pub. No. US 2006/0029795, and/or Pub. No. WO 2006/020619.

The second component 60 of the prosthetic device 10 is configured to secure the first component 50 in a patient's body. As shown in FIG. 1, the second component 60 includes a bearing portion 64 and a fixation portion 62. The bearing portion 64 supports the first component 50, and the fixation portion 62 is adapted to be implanted in or on a bone (e.g., the tibia 30). The fixation portion 62 is configured to be secured or anchored to the bone 30 and is preferably customized to promote bone in-growth.

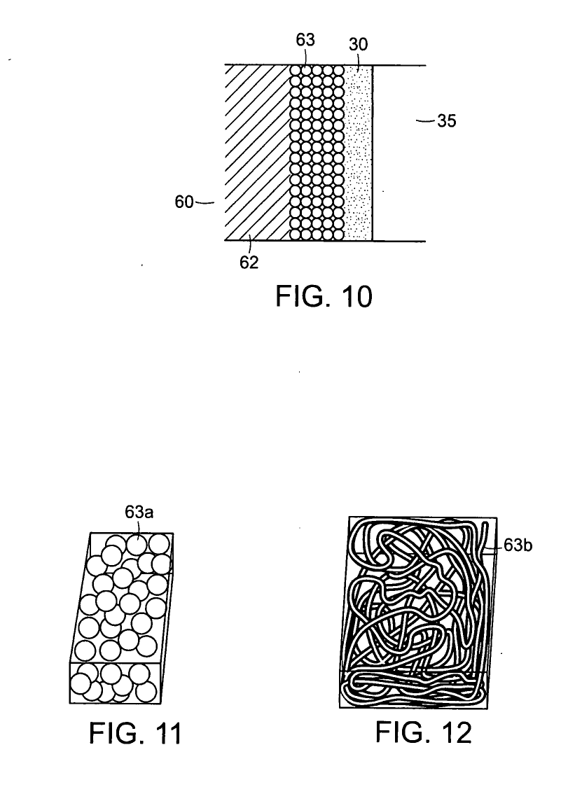

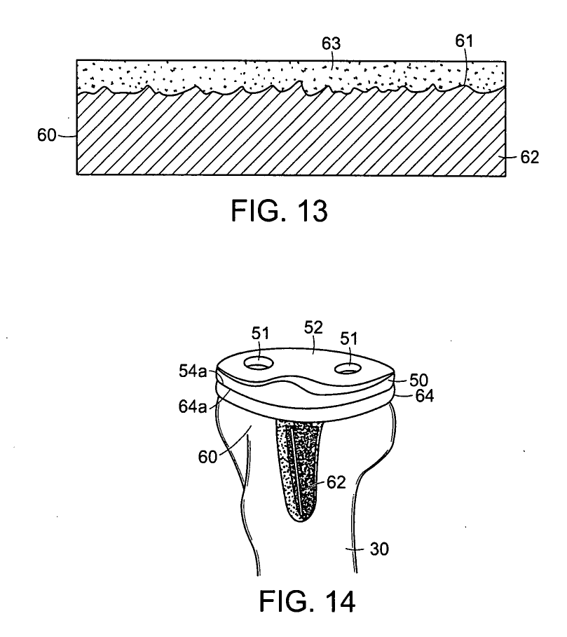

In one embodiment, the fixation portion 62 includes a coating 63 configured for bone in-growth. As shown in FIG. 10, the coating 63 is applied to the fixation portion 62 so that the coating 63 is disposed between the fixation portion 62 and the bone 30 and tissue 35 of the patient. In this manner, the coating 63 provides an interface for bone and/or tissue fixation. The coating 63 may be any known coating suitable for promoting bone in-growth. For example, the coating 63 may be a porous coating, such as a sintered bead coating 63a (FIG. 11), a mesh coating 63b (FIG. 12), or a plasma spray. Additionally or alternatively, the coating 63 may be a bioactive coating, such as hydroxyapatite. In one embodiment, the fixation portion 62 is formed of the polymer composite described in the above referenced Pub. No. US 2006/0030681, Pub. No. US 2006/0029795, and/or Pub. No. WO 2006/020619, and the coating 63 comprises hydroxyapatite. In another embodiment, the fixation portion 62 includes a textured (or roughened) surface 61 (shown in FIG. 13) for bone in-growth, and the coating 63 is a bioactive coating such as hydroxyapatite.

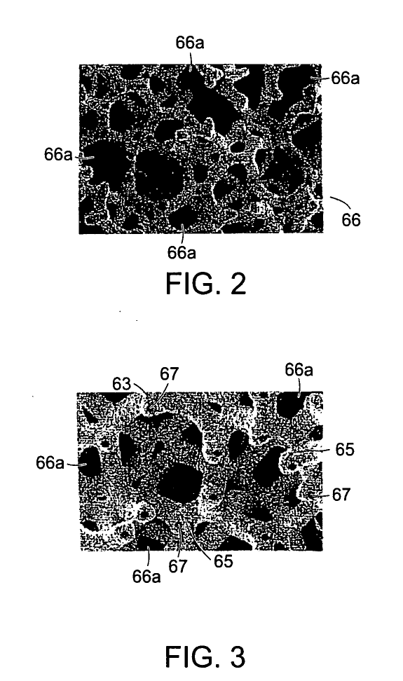

In another embodiment, the fixation portion 62 includes a region of porosity 66 (FIG. 16) that is customized for bone in-growth. For example, as shown in FIG. 2, the region of porosity 66 may include a plurality of cavities (or voids) 66a into which the bone 30 can grow. Preferably, the cavities 66a are formed in sizes and shapes that optimize in-growth. The region of porosity 66 is disposed on the fixation portion 62 so that the region of porosity 66 interfaces with the bone 30 when the prosthetic device 10 is implanted in the patient. For example, as shown in FIG. 16, the region of porosity 66 may be disposed on an outer surface of the fixation portion 62 (and optionally on any part of the bearing portion 64 that interfaces with the bone 30). The region of porosity 66 may be disposed along the entire outer surface of the fixation portion 62 or only at select locations. Additionally, a depth of the region of porosity 66 can be set based on, for example, a desired depth of bone penetration into the fixation portion 62. Alternatively, the region of porosity 62 may encompass the entire fixation portion 62. When the prosthetic device 10 is implanted in the patient's bone 30, the bone 30 will grow into the cavities 66a resulting in fixation of the second component 60 to the bone 30. In this manner, the region of porosity provides an interface for bone fixation.

The region of porosity 66 may take any form that is conducive to bone in-growth. For example, in one embodiment, the region of porosity 66 is made of a metal having an open cellular structure, such as, for example, TRABECULAR METAL™ produced by Zimmer, Inc. and/or a metal as described in U.S. Pat. No. 5,282,861, which is hereby incorporated by reference herein in its entirety. In another embodiment, the region of porosity 66 is made of a metal having an engineered structure, such as, for example, TRABECULITE™ produced by Tecomet and/or a metal as described in U.S. patent application Ser. No. 10/898,659, filed Jul. 23, 2004, Pub. No. US 2005/0112397, which is hereby incorporated by reference herein in its entirety.

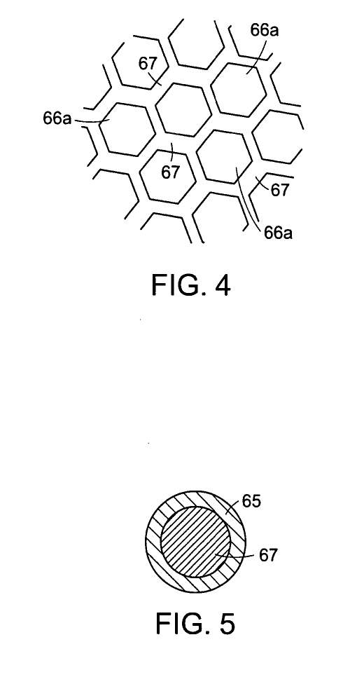

In another embodiment, the region of porosity 66 includes a porous substrate 67 (shown in FIG. 3) that is conducive to bone in-growth. For example, the substrate 67 may have a cellular structure where the cavities 66a are disposed substantially randomly (shown in FIG. 3) or a cellular structure where the cavities 66a are disposed in a substantially predetermined (or engineered) manner (shown in FIG. 4). In one embodiment, the cavities 66a are disposed in a substantially predetermined manner and have a substantially non-homogenous structure. In another embodiment, the substrate 67 is an open cell substrate as described in the above referenced U.S. Pat. No. 5,282,861. The substrate 67 may be made of any material suitable for use in an orthopedic implant, such as a biocompatible metal (e.g., a cobalt-chromium alloy, a titanium alloy, tantalum, or stainless steel); a carbonaceous material (e.g., carbon); a strong ceramic (e.g., an alumina or zirconia-based ceramic); one or more high performance polymers; and/or a polymer composite such as, for example, the polymer composite described in the above referenced Pub. No. US 2006/0030681, Pub. No. US 2006/0029795, and/or Pub. No. WO 2006/020619.

The substrate 67 may optionally include a coating 65, as shown in FIGS. 3 and 5. The coating 65 may be any material suitable for use in an orthopedic implant, such as a biocompatible metal (e.g., a cobalt-chromium alloy, a titanium alloy, tantalum, or stainless steel); a strong ceramic (e.g., an alumina or zirconia-based ceramic); one or more high performance polymers; and/or a polymer composite such as, for example, the polymer composite described in the above referenced Pub. No. US 2006/0030681, Pub. No. US 2006/0029795, and/or Pub. No. WO 2006/020619.

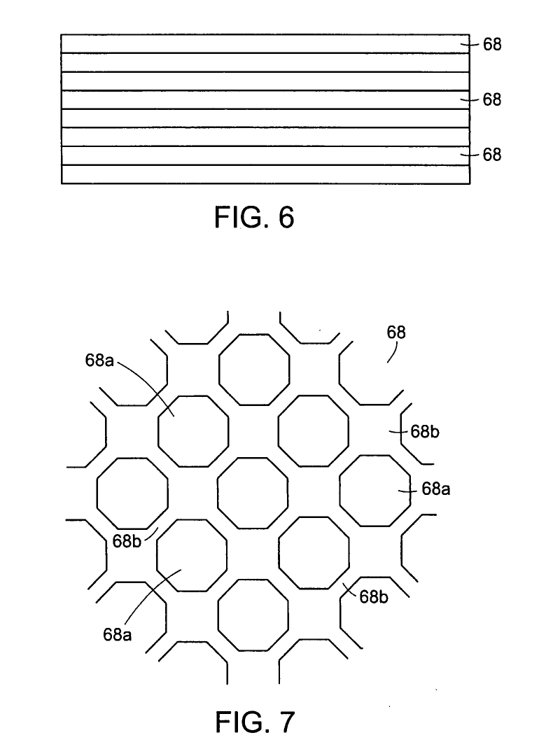

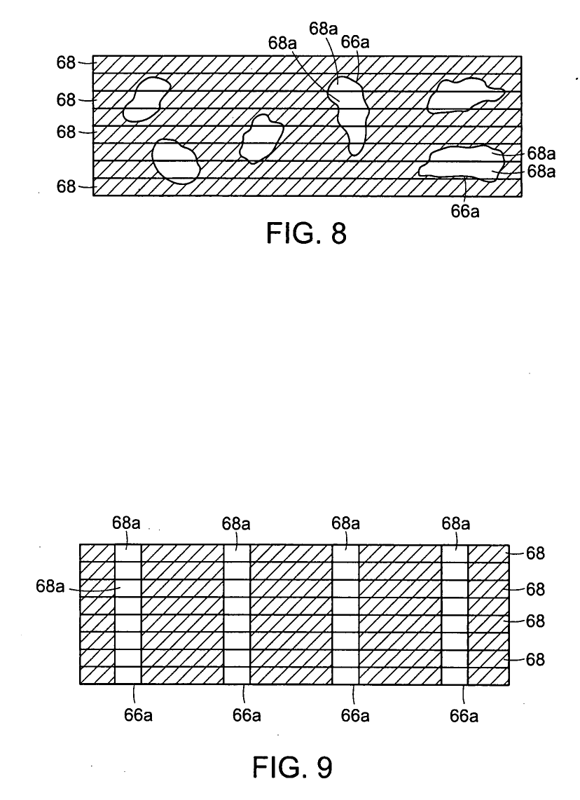

In another embodiment, the region of porosity 66 includes a plurality of layers 68 (FIG. 6) that are conducive to bone in-growth. For example, as shown in FIG. 7, each layer 68 may include a substrate 68b and a plurality of cellular voids 68a. When the layers 68 are assembled (e.g., stacked and bonded together), the cellular voids 68a of one layer 68 combine (e.g., align or overlap) with the cellular voids of adjacent layers 68 to form the cavities 66a. The layers 68 may be oriented so that the cavities 66a formed by the cellular voids 68a are disposed substantially randomly (FIG. 8) and/or in a substantially predetermined (or engineered) manner (FIG. 9). In addition, the layers 68 may be configured so that the region of porosity 66 has varied material properties (e.g., strength, density, modulus of elasticity, stiffness, etc.). For example, one layer 68 may have material properties that are different from material properties of an adjacent layer 68. When the layers 68 are assembled (e.g., stacked and bonded together), the resulting assembly has non-constant or graded material properties. In this manner, the layers 68 can be arranged, for example, to approximate the elastic properties of natural bone and/or to control load distribution and stress concentration. For example, in one embodiment, the layers 68 are configured to transfer at least a portion of a load from the prosthetic device 10 to the bone in a predetermined manner.

The substrate 68b may be made of any material suitable for use in an orthopedic implant, such as a biocompatible metal (e.g., a cobalt-chromium alloy, a titanium alloy, tantalum, or stainless steel); a carbonaceous material (e.g., carbon); a strong ceramic (e.g., an alumina or zirconia-based ceramic); one or more high performance polymers; and/or a polymer composite such as, for example, the polymer composite described in the above referenced Pub. No. US 2006/0030681, Pub. No. US 2006/0029795, and/or Pub. No. WO 2006/020619.

The substrate 68b may optionally include a coating (not shown). The coating may be any material suitable for use in an orthopedic implant, such as a biocompatible metal (e.g., a cobalt-chromium alloy, a titanium alloy, tantalum, or stainless steel); a strong ceramic (e.g., an alumina or zirconia-based ceramic); one or more high performance polymers; and/or a polymer composite such as, for example, the polymer composite described in the above referenced Pub. No. US 2006/0030681, Pub. No. US 2006/0029795, and/or Pub. No. WO 2006/020619. In one embodiment, the coating may include an osteoconductive material that promotes bone in-growth.

In another embodiment, the region of porosity 66 is customized for bone in-growth in a specific joint (e.g., knee, elbow, ankle, shoulder, wrist, spine, and the like) and may be configured for bone in-growth of a predetermined type of bone (e.g., cancellous bone, cortical bone). In this embodiment, a configuration of the region of porosity 66 is determined based on characteristics of the specific joint in which the prosthetic device 10 will be implanted. Thus, if the prosthetic device 10 is a knee implant, characteristics of the knee bone (e.g., femur, tibia, patella) on which the prosthetic device 10 will be implanted will be accounted for in the design of the prosthetic device 10. For example, the region of porosity 66 may have a microstructure that is similar to a microstructure of natural bone (e.g., cancellous bone, cortical bone) in the specific joint. This can be accomplished, for example, by forming the region of porosity 66 of TRABECULAR METAL™, which is produced by Zimmer, Inc., and/or of the material described in the above referenced U.S. Pat. No. 5,282,861. Similarly, a porosity of the region of porosity 66 (i.e., a volume percent of the cavities 66a in the region of porosity 66) may be similar to a porosity of the bone 30 on which the implant 12 will be affixed. The porosity may be constant or may vary through the region of porosity 66. The porosity may also be customized based on various factors, such as rate of growth of the bone 30, a desired degree of penetration (or infiltration) of the bone 30 into the fixation portion 62, and the like. In one embodiment, the porosity of the region of porosity 66 may be in a range of between about 60 percent to about 90 percent. In another embodiment, the porosity may be in a range of between about 75 percent to about 80 percent. In other embodiments, the porosity may be approximately 70 percent or approximately 80 percent. Additionally, a location and/or extent of the region of porosity 66 on the fixation portion 62 may be strategically determined based on factors, such as a desired load distribution in the joint, kinetics, and/or a degree of bone infiltration required for adequate fixation of the bone 30 to the fixation portion 62. In this manner, the fixation portion 62 may be customized and/or optimized for bone in-growth in a specific joint (e.g., designed to achieve adequate bone in-growth for fixation in minimal time).

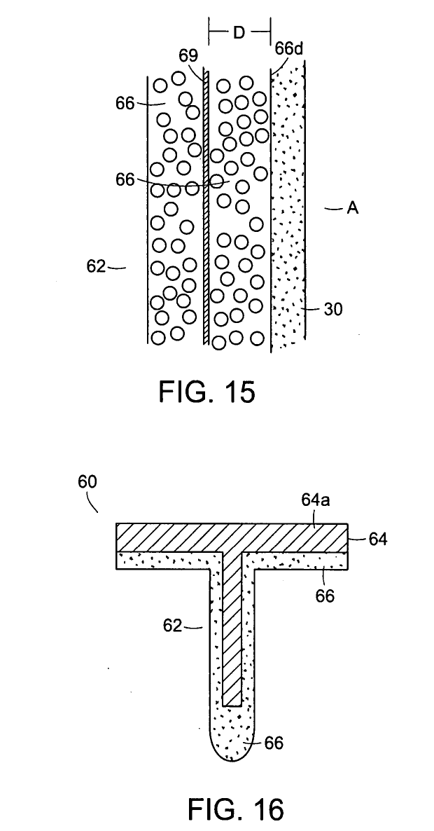

In another embodiment, the fixation portion 62 is configured to limit the degree of infiltration of the bone 30 into the fixation portion 62. This may be desirable, for example, in applications where future removal of the implant 12 is contemplated and where overgrowth of the bone 30 into the implant 12 would make such removal difficult and/or traumatic. Overgrowth may be limited, for example, by setting a depth of the region of porosity 66 at approximately a desired depth D of bone infiltration. Alternatively, as shown in FIG. 15, the region of porosity 66 may include a barrier layer 69 disposed at the depth D. The barrier layer 69 (which may be a single layer or multiple layers) is configured to substantially prevent and/or slow progression of the bone 30 in a direction A (e.g., a direction into the region of porosity 66 from an outer surface 66d of the region of porosity 66). The barrier layer 69 may be a solid layer and/or a layer having a porosity less than the porosity of the region of porosity 66. The barrier layer 69 may be continuous or disposed discretely at strategic locations in the region of porosity 66. In addition to limiting infiltration of the bone 30 into the fixation portion 62, the barrier layer 69 may be incorporated into any porous region of the implant 12 to limit a depth of penetration of a polymer or polymer composite when the polymer or polymer composite is molded to the porous region.

The barrier layer 69 may be made of any material suitable for use in orthopedic implant applications. For example, the barrier layer 69 may be a biocompatible metal (e.g., a cobalt-chromium alloy, a titanium alloy, tantalum, or stainless steel); a strong ceramic (e.g., an alumina or zirconia-based ceramic); one or more high performance polymers, and/or a polymer composite such as, for example, the polymer composite described in the above referenced Pub. No. US 2006/0030681, Pub. No. US 2006/0029795, and/or Pub. No. WO 2006/020619.

The second component 60 may be made of any material (or combination of materials) suitable for use in orthopedic implant applications. For example, the second component 60 may include a biocompatible metal (e.g., a cobalt-chromium alloy, a titanium alloy, tantalum, or stainless steel); a carbonaceous material (e.g., carbon); a strong ceramic (e.g., an alumina or zirconia-based ceramic); one or more high performance polymers, and/or a polymer composite such as, for example, the polymer composite described in the above referenced Pub. No. US 2006/0030681, Pub. No. US 2006/0029795, and/or Pub. No. WO 2006/020619.

In regard to material selection, the second component 60 is preferably configured to have material properties (e.g., strength, density, modulus of elasticity, stiffness, weight, load distribution) that approximate those of natural bone, such as cancellous bone and/or cortical bone, a vertebral body, and the like. This enables the second component 60 to closely approximate the natural behavior of the joint in which the second component 60 is implanted. For example, in one embodiment, at least a portion of the second component 60 has a microstructure similar to a microstructure of the bone 30 on which the second component 60 is implanted. This can be accomplished, for example, by forming a portion of the second component 60 of TRABECULAR METAL™, which is produced by Zimmer, Inc., and/or of the material described in the above referenced U.S. Pat. No. 5,282,861. In another embodiment, at least a portion of the second component 60 is formed of the polymer composite described in the above referenced Pub. No. US 2006/0030681, Pub. No. US 2006/0029795, and/or Pub. No. WO 2006/020619. The polymer composite is graded so that a modulus of elasticity of a portion of the second component 60 is similar to a modulus of elasticity of the bone 30.

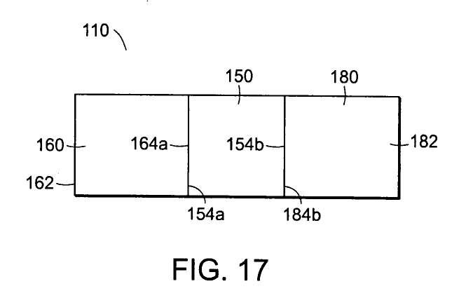

FIG. 17 shows a prosthetic device 110 according to the present invention. The prosthetic device 110 is similar to the implant 12 of the prosthetic device 10 but is adapted for use as a spine implant. The prosthetic device 110 includes a first component 150, a second component 160, and a third component 180. When the prosthetic device 110 is implanted in a spine of a patient, the first component 150 replaces an intervertebral disc and the second and third components 160 and 180 are attached or cemented to vertebral bodies.

The first component 150 of the prosthetic device 110 is similar to the first component 50 of the implant 12 except the first component 150 is adapted to be disposed between the second component 160 and the third component 180. For example, the first component 150 may include (a) a first surface 154a configured to interface with a surface 164a of the second component 160 and (b) a second surface 154b configured to interface with a surface 184b of the third component 180. As with the components of the implant 12, the first component 150 may be secured to the second component 160 and/or to the third component 180 by mechanical devices or joints, bonding, molding, chemical devices or joints, and/or adhesive. The first component 150 may also be integral with the second component 160 and/or the third component 180. In a preferred embodiment, the first component 150 includes the polymer composite described in the above referenced Pub. No. US 2006/0030681, Pub. No. US 2006/0029795, and/or Pub. No. WO 2006/020619, and the polymer composite is graded so that a modulus of elasticity of the first component is similar to a modulus of elasticity of an intervertebral disc, as described, for example, in the above referenced Pub. No. US 2006/0029795.

The second component 160 and the third component 180 of the prosthetic device 110 are similar to the second component 60 of the implant 12. For example, the second and third components 160 and 180 include fixation portions 162 and 182, respectively. The fixation portions 162 and 182 are equivalent to the fixation portion 62 of the implant 12. In a preferred embodiment, the second component 160 and/or the third component 180 includes the polymer composite described in the above referenced Pub. No. US 2006/0030681, Pub. No. US 2006/0029795, and/or Pub. No. WO 2006/020619, and the polymer composite is graded so that a modulus of elasticity of the second component 160 and/or the third component 180 is similar to a modulus of elasticity of a vertebral body, as described, for example, in the above referenced Pub. No. US 2006/0029795. In another embodiment, the second component 160 and/or the third component 180 has a microstructure similar to a microstructure of a vertebral body.

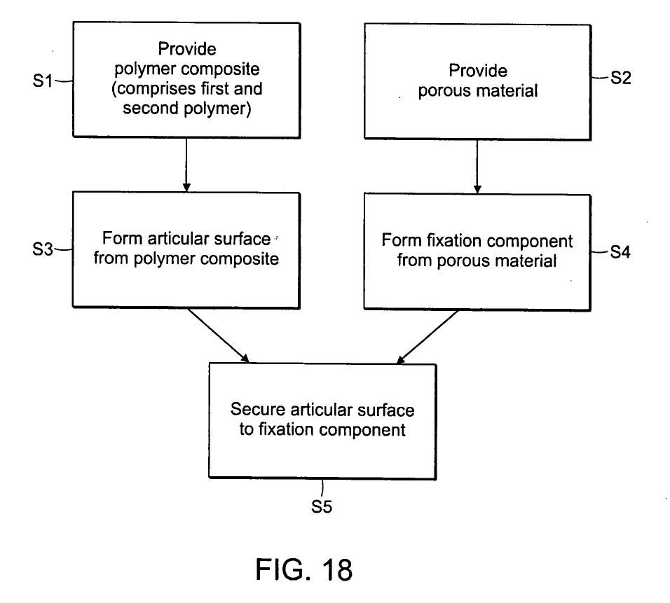

FIG. 18 shows a method of making a prosthetic device according to the present invention that incorporates the following steps. In step S1, a polymer composite having a wear rate of less than approximately 10⁻⁷ mm³/Nm is provided. The polymer composite includes a first polymer and a second polymer, and the first polymer comprises at least 10 weight percent of the polymer composite. In step S2, a porous material is provided. A porosity of the porous material is determined based on characteristics of a bone of a specific joint. In step S3, the polymer composite is formed into an articular surface of the prosthetic device. In step S4, the porous material is formed into a fixation component that is configured to be implanted on the bone. In step S5, the articular surface is secured (or connected) to the fixation component. The articular surface may be secured directly to the fixation component or may be secured to an intermediate component that is secured to the fixation component.

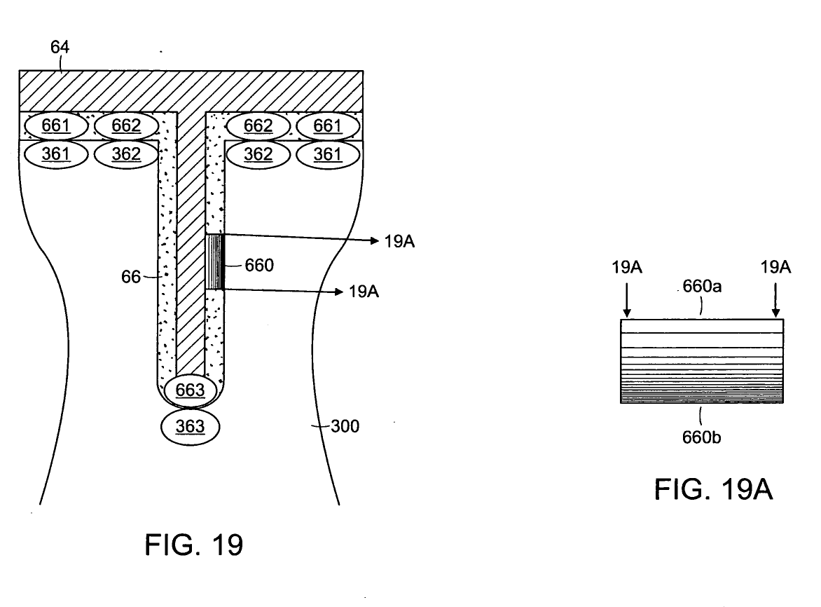

Turning now to FIG. 19, it shows an example of how a region of porosity 66 in the second component 60 may be configured to approximate the properties of natural bone (and thus allow and promote the in-growth into the second component of natural bone of a patient) and/or control load distribution and stress concentration according to one embodiment of the invention. The sample characteristics of the regions of the natural bone 300 are such that the outer bone region 361, made of cortical bone, is hard and dense, whereas the inner bone region 362 and innermost bone region 363, made of cancellous bone, are foam-like. As described in the above referenced U.S. Pat. No. 5,282,861, materials used for prosthetic devices should be engineered to closely mimic the properties of natural bone. In doing so, the prosthetic device can more properly distribute stresses throughout the structure, the in-growing bone, and the surrounding bone to avoid resorption and weakening caused by load stress. Thus, the inner regions of porosity 662 and innermost region of porosity 663 can be customized to have a larger depth of porosity in order to promote bone in-growth and provide elasticity to facilitate transfer of sheer loads to the outer layers of the cortical bone with the inner bone portion 362 and the innermost bone region 363, respectively. Similarly, the outer region of porosity 661 can be customized to be more dense with a lower depth of porosity in order to better bear load and stress as it comes into contact with the outer bone region 361 of the natural bone 300. The innermost region or porosity 663 could further be customized to have an even greater depth of porosity as it must facilitate the highest bone in-growth with the corresponding innermost bone region 363 deep within the natural bone 300. FIG. 19 also shows an example of a depth perspective view of a sample portion 660 of the region of porosity 66. As disclosed within the above referenced Pub. No. US 2005/0112397, a sample portion 660 of a region of porosity 66 can be comprised of stacked and bonded sheets. A graduated level of porosity may therefore be constructed from the inner surface 660a to an inner outer layer 660b as desired to achieve higher or lower designed-for customized bone in-growth and/or load distribution as discussed above.

Thus, according to embodiments of the present invention, a prosthetic device having improved wear resistance, elastic properties, fixation, and longevity is provided.

Other embodiments of the invention will be apparent to those skilled in the art from consideration of the specification and practice of the invention disclosed herein. It is intended that the specification and examples be considered as exemplary only.

What is claimed is:

1. A prosthetic device comprising:
a first component including a polymer composite configured as an articular surface, the polymer composite including a first polymer and a second polymer,
the first polymer comprising at least 10 weight percent of the polymer composite and a wear rate of the polymer composite being less than approximately 10⁻⁷ mm³/Nm, and a second component configured to be implanted on a bone, the second component including a region of porosity that is customized for bone in-growth in a specific joint of a patient.

2. The prosthetic device of claim 1, wherein the porosity is configured for bone in-growth of a predetermined type of bone.

3. The prosthetic device of claim 1, wherein the first polymer is configured to promote articulation.

4. The prosthetic device of claim 1, wherein the first polymer is configured to reduce at least one of friction and wear during articulation.

5. The prosthetic device of claim 1, wherein the second polymer is configured to promote bone in-growth.

6. The prosthetic device of claim 1, where the first polymer is selected from the group consisting of PTFE, ultra-high molecular weight polyethylene, and nylon.

7. The prosthetic device of claim 1, wherein the second polymer is selected from the group consisting of PEEK, polyaryletherketone, polyimide, nylon, polycarbonate, acrylonitrile, ABS, and ultra-high molecular weight polyethylene.

8. The prosthetic device of claim 1, wherein the second polymer comprises between about 10 weight percent and about 90 weight percent of the polymer composite.

9. The prosthetic device of claim 8, wherein the second polymer comprises about 50 weight percent or less of the polymer composite.

10. The prosthetic device of claim 1, wherein a modulus of elasticity of at least a portion of the first component is similar to a modulus of elasticity of cartilage.

11. The prosthetic device of claim 1, wherein a modulus of elasticity of at least a portion of the second component is similar to a modulus of elasticity of at least one of cancellous bone and cortical bone.

12. The prosthetic device of claim 1, wherein the region of porosity of the second component has a microstructure similar to a microstructure of at least one of cancellous bone and cortical bone.

13. The prosthetic device of claim 1, wherein the region of porosity of the second component includes a porous substrate.

14. The prosthetic device of claim 13, wherein the porous substrate includes a cellular structure that is substantially random.

15. The prosthetic device of claim 13, wherein the porous substrate includes a cellular structure that is substantially predetermined.

16. The prosthetic device of claim 13, wherein the porous substrate includes a cellular structure that is substantially predetermined and has a substantially non-homogenous structure.

17. The prosthetic device of claim 13, wherein the porous substrate comprises at least one of a metal, a polymer, a ceramic, and a carbon.

18. The prosthetic device of claim 17, wherein the metal is selected from the group consisting of tantalum, titanium, cobalt chrome, stainless steel, and zirconium.

19. The prosthetic device of claim 13, wherein the porous substrate includes a coating.

20. The prosthetic device of claim 19, wherein the coating includes at least one of a metal, a polymer, a ceramic, and a material configured to promote bone in-growth.

21. The prosthetic device of claim 20, wherein the metal is selected from the group consisting of tantalum, titanium, cobalt chrome, stainless steel, and zirconium.

22. The prosthetic device of claim 1, wherein the region of porosity of the second component comprises a plurality of layers.

23. The prosthetic device of claim 22, wherein each layer includes a plurality of cellular voids.

24. The prosthetic device of claim 23, wherein the layers are configured so that the cellular voids are oriented substantially randomly.

25. The prosthetic device of claim 23, wherein the layers are configured so that the cellular voids are oriented in a substantially predetermined manner.

26. The prosthetic device of claim 23, wherein the layers are configured so that the cellular voids are oriented in a substantially predetermined manner and have a substantially non-homogenous structure.

27. The prosthetic device of claim 22, wherein the layers are configured to transfer at least a portion of a load from the prosthetic device to the bone in a predetermined manner.

28. The prosthetic device of claim 22, wherein at least one of the layers comprises at least one of a metal, a polymer, and a ceramic.

29. The prosthetic device of claim 22, wherein the layers are joined by at least one of bonding, a mechanical joint, a chemical joint, and an adhesive.

30. The prosthetic device of claim 22, wherein the plurality of layers includes a first layer and a second layer, the second layer including at least one elastic property that is different from an elastic property of the first layer.

31. The prosthetic device of claim 30, wherein first and second layers are arranged to control stress concentration in the bone.

32. The prosthetic device of claim 1, wherein the region of porosity of the second component includes a porosity of between about 60 percent to about 90 percent.

33. The prosthetic device of claim 32, wherein the region of porosity of the second component includes a porosity of between about 75 percent to about 85 percent.

34. The prosthetic device of claim 32, wherein the region of porosity of the second component includes a porosity of approximately 70 percent.

35. The prosthetic device of claim 32, wherein the region of porosity of the second component includes a porosity of approximately 80 percent.

36. The prosthetic device of claim 1, wherein the second component includes at least one of a porous coating and a bioactive coating.

37. The prosthetic device of claim 36, wherein the porous coating is selected from the group consisting of beads, plasma spray, and mesh.

38. The prosthetic device of claim 36, wherein the bioactive coating comprises hydroxyapatite.

39. The prosthetic device of claim 1, wherein at least a portion of the second component includes the polymer composite.

40. The prosthetic device of claim 39, wherein at least a portion of the polymer composite in the second component is coated with a bioactive coating.

41. The prosthetic device of claim 1, wherein the second component includes a textured surface.

42. The prosthetic device of claim 41, wherein the textured surface is coated with a bioactive coating.

43. The prosthetic device of claim 1, wherein the first component is configured to be attached to a plurality of biological cells.

44. The prosthetic device of claim 43, wherein the cells comprise at least one of cartilage cells and stem cells.

45. The prosthetic device of claim 1, wherein the first and second components are configured to be bonded together.

46. The prosthetic device of claim 1, wherein the first and second components are configured to be molded together.

47. The prosthetic device of claim 1, wherein the region of porosity of the second component is configured to limit a depth of penetration of the polymer composite when the first component and the second component are molded together.

48. The prosthetic device of claim 1, wherein the first component is integral with the second component.

49. The prosthetic device of claim 1, wherein the second component includes a first surface configured to interface with the bone and a second surface configured to interface with the first component.

50. The prosthetic device of claim 1, wherein the second component is configured to limit a depth of bone in-growth into the region of porosity.

51. The prosthetic device of claim 1, wherein the prosthetic device is configured as a joint implant.

52. The prosthetic device of claim 51, wherein the joint implant is a total joint implant.

53. The prosthetic device of claim 51, wherein the joint implant is a partial joint implant.

54. The prosthetic device of claim 51, wherein the joint implant is a local defect implant.

55. The prosthetic device of claim 1, further comprising a third component configured to be implanted on a bone, wherein the third component includes a region of porosity that is customized for bone in-growth in the specific joint.

56. The prosthetic device of claim 55, wherein the first component is configured to be disposed between the second component and the third component when the prosthetic device is implanted on a spine of a patient.

57. The prosthetic device of claim 56, wherein a modulus of elasticity of at least a portion of the first component is similar to a modulus of elasticity of an intervertebral disc.

58. The prosthetic device of claim 56, wherein a modulus of elasticity of at least a portion of the second component and/or the third component is similar to a modulus of elasticity of a vertebral body.

59. A prosthetic device for implantation in a patient, comprising:
a bearing surface comprising a polymer composite including a first polymer and a second polymer and having a wear rate that is less than approximately 10⁻⁷ mm³/Nm, the first polymer being at least 10 weight percent of the polymer composite, and
a fixation portion including a plurality of cavities, the volume percent of the cavities in the fixation portion being determined based on one or more aspects of a specific joint of the patient.

60. The prosthetic device of claim 59, wherein the volume percent is determined based on at least one of bone structure of the specific joint and one or more growth characteristics of the specific joint.

61. A method of making a prosthetic device, comprising:
providing a polymer composite that includes a first polymer and a second polymer, wherein the first polymer comprises at least 10 weight percent of the polymer composite and a wear rate of the polymer composite is less than approximately 10⁻⁷ mm³/Nm,
providing a porous material, wherein a porosity of the porous material is determined based on characteristics of a bone of a specific joint of a patient,
forming an articular surface from the polymer composite,
forming a fixation component from the porous material, wherein the fixation component is configured to be implanted on the bone, and
connecting the articular surface to the fixation component.

62. The method of claim 61, wherein the step of connecting the articular surface to the fixation component includes connecting the articular surface to a first side of an intermediate component and connecting the fixation component to a second side of the intermediate component.
